SEQUENCE LISTING (10 sequences)

SEQ ID NO: 1 - protein, 10 aa, synthetic construct
GDSITNGFWI

SEQ ID NO: 2 - protein, 9 aa, synthetic construct
YISYSGSTY

SEQ ID NO: 3 - protein, 13 aa, synthetic construct
YRSYGRTPYYFDY

SEQ ID NO: 4 - protein, 11 aa, synthetic construct
KASSNLGHAVA

SEQ ID NO: 5 - protein, 7 aa, synthetic construct
SASNRYT

SEQ ID NO: 6 - protein, 9 aa, synthetic construct
QQYDDYPYT

SEQ ID NO: 7 - protein, 120 aa, synthetic construct
EVQLQESGPGLVKPSETLSLTCTVSGDSITNGFWIWIRKPPGNKLEYMGYISYSGSTYYNPSLKSRISISRDTSKNQFSLKLSSVTAADTGVYYCAYRSYGRTPYYFDYWGQGTTLTVSS

SEQ ID NO: 8 - protein, 107 aa, synthetic construct
DIVMTQSPSFLSASVGDRVTITCKASSNLGHAVAWYQQKPGKSPKLLIYSASNRYTGVPDRFSGSGSGTDFTLTISSLQPEDFADYFCQQYDDYPYTFGGGTKLEIK

SEQ ID NO: 9 - protein, 450 aa, synthetic construct
EVQLQESGPGLVKPSETLSLTCTVSGDSITNGFWIWIRKPPGNKLEYMGYISYSGSTYYNPSLKSRISISRDTSKNQFSLKLSSVTAADTGVYYCAYRSYGRTPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIVMTQSPSFLSASVGDRVTITCKASSNLGHAVAWYQQKPGKSPKLLIYSASNRYTGVPDRFSGSGSGTDFTLTISSLQPEDFADYFCQQYDDYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0392022 A1
(43) Pub. Date: Nov. 28, 2024

(54) TREATMENT OF MULTIPLE SCLEROSIS WITH ANTI-CD40L ANTIBODIES

(71) Applicant: Genzyme Corporation, Cambridge, MA (US)

(72) Inventors: Christine FARENC, Paris (FR); Philippe TRUFFINET, Paris (FR); Fabienne SCHUMACHER, Frankfurt (DE)

(73) Assignee: Genzyme Corporation, Cambridge, MA (US)

(21) Appl. No.: 18/671,636

(22) Filed: May 22, 2024

(30) Foreign Application Priority Data
May 22, 2023 (EP) 23174655.3
May 22, 2023 (EP) 23174657.9

(51) Int. Cl. C07K 16/28; A61K 39/00; A61P 25/28

(52) U.S. Cl. CPC C07K 16/2875; A61P 25/28; A61K 2039/505; C07K 2317/21

(57) ABSTRACT

The present invention relates to treatment of relapsing forms of multiple sclerosis using an anti-human CD40L monoclonal antibody.

### CROSS-REFERENCE TO RELATED APPLICATION

This application claims priority to European Patent Application 23174655.3 and European Patent Application 23174657.9, both filed May 22, 2023. The disclosures of those priority applications are incorporated by reference herein in their entirety.

### SEQUENCE LISTING

The instant application contains a Sequence Listing which has been submitted electronically in XML format and is hereby incorporated by reference herein in its entirety. The electronic copy of the Sequence Listing, created on May 16, 2024, is named 022548.US033_SL.xml and is 10,366 bytes in size.

### BACKGROUND OF THE INVENTION

Multiple sclerosis (MS) is a chronic, immune-mediated inflammatory and neurodegenerative disease that affects the central nervous system. It is characterized by loss of motor and sensory function resulting from inflammation, demyelination, and axonal injury and loss (Friese et al., Nat Rev Neurol. (2014) 10 (4): 225-38; Trapp and Nave, Ann Rev Neurosci. (2008) 231:247-69). MS affects more than two million people worldwide and is at least two to three times more prevalent in women than in men. MS patients display a wide range of severe clinical symptoms with increased physical disability, fatigue, pain, and cognitive impairment as the disease progresses. It has a significant impact on patients' quality of life and shortens patients' life expectancy by five to ten years on average.

Immunomodulatory drugs are the mainstay of MS therapy. Beyond the existing strategy of modulating the cellular elements of adaptive immunity, there is mounting evidence that innate immunity is responsible for many of the neurodegenerative aspects of MS that persist despite the use of approved disease-modifying therapies to prevent acute relapses. Even the most recent therapies act mainly on adaptive immunity in the periphery with only modest or temporary ability to halt neuroinflammatory and neurodegenerative processes and stop disease progression. There is still a significant unmet need for therapies that target neuroinflammation in the CNS with a goal of halting long-term disability and neurodegeneration in people with relapsing MS as well as in those with progressive forms of MS. Therefore, development of MS treatments with new modes of action involving not only adaptive but also innate immunity is of interest.

Human cluster of differentiation 40 ligand (CD40L), also known as CD154, is a TNF family member that is primarily expressed on activated T cells and binds to CD40 on antigen-presenting cells. The CD40/CD40L costimulatory pathway is critical for humoral immune response, as well as for proinflammatory cytokine secretion by macrophages and reciprocal costimulation between T lymphocytes and antigen-presenting cells. Disruption of the CD40/CD40L pathway in autoimmune diseases, particularly those such as MS where pathogenic B cell responses are a key hallmark of disease, may impact both cellular and humoral responses and have therapeutic benefit.

The importance of the CD40/CD40L pathway in MS immune signaling is indirectly supported by observed increase of soluble CD40L in people with MS. Higher levels of soluble CD40L are observed in people with RRMS as compared to patients with SPMS, relating to an increase of CD40L expression on immune cells and suggesting different involvement of this pathway in the two MS types.

In view of the medical needs that targeting CD40L may address, there is a need for improved MS therapies that target CD40L.

### SUMMARY OF THE INVENTION

The present disclosure provides methods for MS therapy using anti-human CD40L antibody frexalimab or a related antibody.

In some embodiments, the present disclosure provides a method of treating relapsing multiple sclerosis (MS) in a human patient in need thereof. The relapsing MS may be, e.g., relapsing-remitting MS or secondary progressive MS with relapses.

In certain embodiments, the treatment comprises intravenously administering to the patient a monoclonal anti-human CD40L antibody, wherein the heavy chain CDR1-3 and light chain CDR1-3 of said antibody comprise SEQ ID NOs: 1-6, respectively, and wherein the antibody is administered to the patient at a dose of 1000-2000 mg (e.g., every four weeks or about every month). In some embodiments, the dose is 1200 mg. In certain embodiments, the first administration of the antibody is at a first dose (e.g., a “loading dose”), and subsequent administration is at a second dose. The first dose may be higher than the second dose. For example, in certain embodiments, the first dose may be 1800 mg and/or the second dose may be 1200 mg.

In certain embodiments, the treatment comprises administering to the patient a monoclonal anti-human CD40L antibody, wherein the heavy chain CDR1-3 and light chain CDR1-3 of said antibody comprise SEQ ID NOs: 1-6, respectively, and wherein the antibody is administered to the patient at a dose of 1800 mg (e.g., about every four weeks or about every month). In particular embodiments, the antibody is administered subcutaneously. In some embodiments, the first administration of the antibody is by one route and subsequent administration is by other route(s). For example, the first administration of the antibody may be intravenous and subsequent administration may be subcutaneous.

In some embodiments, the antibody is of isotype subtype IgG₁.

In some embodiments, the antibody comprises a heavy chain variable domain and a light chain variable domain comprising SEQ ID NOs: 7 and 8, respectively.

In some embodiments, the antibody comprises a heavy chain and a light chain comprising SEQ ID NOs: 9 and 10, respectively.

In some embodiments, the present disclosure provides a method of treating relapsing MS in a human patient in need thereof, comprising administering to the patient by intravenous infusion a monoclonal anti-human CD40L antibody that comprises a heavy chain and a light chain comprising SEQ ID NOs: 9 and 10, respectively, wherein the antibody is administered at a first dose of 1800 mg and at subsequent doses of 1200 mg, wherein the interval between doses is about four weeks or about one month.

In some embodiments, the present disclosure provides a method of treating relapsing MS in a human patient in need thereof, comprising administering to the patient a monoclonal anti-human CD40L antibody that comprises a heavy chain and a light chain comprising SEQ ID NOs: 9 and 10, respectively, wherein the antibody is administered at a dose of 1800 mg, wherein the first dose is administered intravenously and subsequent doses are administered subcutaneously, and wherein the interval between doses is about four weeks or about one month.

The present disclosure also provides a monoclonal anti-human CD40L antibody recited herein for use in treating a human patient in a therapy recited herein, and use of a monoclonal anti-human CD40L antibody recited herein for the manufacture of a medicament for treating a human patient in a therapy recited herein. Kits and articles of manufacture comprising the recited anti-human CD40L antibodies (e.g., for use in a therapy recited herein) are also provided.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### DETAILED DESCRIPTION OF THE INVENTION

The present disclosure provides safe and efficacious treatment of relapsing MS (RMS) with frexalimab or a related antibody (such as an antibody described herein, e.g., an antibody having the same heavy and light chain CDRs, or the same heavy and light chain variable domains, as frexalimab). Frexalimab is a humanized anti-human CD40L IgG₁ antibody that has high affinity for human CD40L, induces immune tolerance, and blocks humoral immunity, without eliciting thrombogenic or clotting reactions. See also U.S. Pat. No. 10,874,738 B2, the disclosure of which is incorporated by reference herein in its entirety.

The frexalimab heavy chain sequence (SEQ ID NO: 9) is shown below, with its variable domain sequence in boldface and italics (SEQ ID NO: 7) and its CDR1-3 (SEQ ID NOs: 1-3, respectively) underlined:

(SEQ ID NO: 9)
EVQLQESGPGLVKPSETLSLTCTVSGDSITNGEWIWIRKPPGNKL
EYMGYISYSGSTYYNPSLKSRISISRDTSKNQFSLKLSSVTAADT
GVYYCAYRSYGRTPYYFDYWGQGTTLTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKAEPKSCDK
THTCPPCPAPELLGGPSVELFPPKPKDTLMISRTPEVTCVVVDVS
HRDPEVKENWYVDGVEVHNAKTKPREEQYNSTYRVVSVLIVLHQD
WLNGKEYKCAVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK

The frexalimab light chain sequence (SEQ ID NO: 10) is shown below, with its variable domain sequence in boldface and italics (SEQ ID NO: 8) and its CDR1-3 (SEQ ID NOs: 4-6, respectively) underlined:

(SEQ ID NO: 10)
DIVMTQSPSFLSASVGDRVTITCKASSNLGHAVAWYQQKPGKSPK
LLIYSASNRYTGVPDRFSGSGSGTDFTLTISSLQPEDFADYFCQQ
YDDYPYTEGGGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

In some embodiments, an MS therapy described herein uses an anti-CD40L antibody that is frexalimab or a related antibody (e.g., an antibody described herein), or an antigen-binding portion of said anti-CD40L antibody. In some embodiments, the anti-CD40L antibody comprises the six CDR amino acid sequences of frexalimab. The CDRs may be assigned, e.g., in accordance with IMGT® definitions (Lefranc et al., Dev Comp Immunol. (2003) 27 (1): 55-77); or in accordance with the definitions of Kabat, Sequences of Proteins of Immunological Interest (National Institutes of Health, Bethesda, MD (1987 and 1991)); Chothia & Lesk, J Mol Biol. (1987) 196:901-17; Chothia et al., Nature (1989) 342:878-83; MacCallum et al., J Mol Biol. (1996) 262:732-45; or Honegger and Plückthun, J Mol Biol. (2001) 309 (3): 657-70. In certain embodiments, the anti-CD40L antibody comprises H-CDR1-3 and L-CDR1-3 amino acid sequences of SEQ ID NOs: 1-6, respectively.

In some embodiments, the anti-CD40L antibody may comprise a heavy chain variable domain (VH) that is at least 90% (e.g., at least 95%, 98%, or 99%) identical in sequence to SEQ ID NO: 7, and a light chain variable domain (VL) that is at least 90% (e.g., at least 95%, 98%, or 99%) identical in sequence to SEQ ID NO: 8. Optionally, said antibody may comprise the six CDRs of frexalimab. In some embodiments, the anti-CD40L antibody comprises the heavy and light chain variable domain amino acid sequences of frexalimab. In certain embodiments, the anti-CD40L antibody comprises a VH comprising SEQ ID NO: 7 and a VL comprising SEQ ID NO: 8.

In some embodiments, the anti-CD40L antibody comprises a heavy chain (HC) that is at least 90% (e.g., at least 95%, 98%, or 99%) identical in sequence to SEQ ID NO: 9, and a light chain (LC) that is at least 90% (e.g., at least 95%, 98%, or 99%) identical in sequence to SEQ ID NO: 10. Optionally, said antibody may comprise the six CDRs or the VH and VL of frexalimab. In certain embodiments, the anti-CD40L antibody comprises an HC comprising SEQ ID NO: 9 and an LC comprising SEQ ID NO: 10.

Frexalimab and related antibodies as described herein can be expressed in, for example, mammalian host cells such as CHO cells, NS0 cells, COS cells, 293 cells, and SP2/0 cells. In some embodiments, the C-terminal lysine of the heavy chain of the antibody is removed. The antibody can be provided, for example, in powder form (e.g., lyophilized form) that is reconstituted in a suitable pharmaceutical solution before administration to a patient, or in an aqueous pharmaceutical solution. In certain embodiments, the antibody is provided in a pharmaceutical composition that further comprises L-histidine HCl, L-histidine, L-arginine HCl, sucrose, polysorbate-80, and water. In particular embodiments, the pH of the pharmaceutical composition is 5.6.

In some embodiments, a pharmaceutical composition comprising an anti-CD40L antibody as recited herein (e.g., frexalimab) is provided in an article of manufacture or kit such as one that comprises one or more containers containing the composition and a label associated with the container(s). The container may be a single use container (e.g., for intravenous or subcutaneous delivery), such as a single use boule or vial, or a single use, pre-filled syringe or injector. In some embodiments, the container contains a single dose (e.g., a dose recited herein) of the anti-CD40L antibody, wherein the container may be a vial or a pre-filled syringe or injector.

In some embodiments, the article of manufacture or kit contains a first container and subsequent container(s), wherein the first container contains a different (e.g., higher) dose of the antibody than the subsequent containers. For example, the first container may contain a single dose of the anti-CD40L antibody (e.g., frexalimab) in an amount of 1800 mg, while the subsequent container(s) may contain single dose(s) of the same antibody in an amount of 1200 mg, e.g., wherein the container may be designed for intravenous administration of the antibody.

In some embodiments, the article of manufacture or kit contains containers that each contain a single dose of the anti-CD40L antibody (e.g., frexalimab) in an amount of 1800 mg, e.g., wherein the containers are designed for subcutaneous administration of the antibody.

In some embodiments, the article of manufacture or kit contains a first container and subsequent container(s), wherein the first and subsequent containers are designed for different routes of administration (e.g., intravenous and subcutaneous). For example, the first container may contain a single dose of the anti-CD40L antibody (e.g., frexalimab) in an amount of 1800 mg and be designed for intravenous administration of the antibody, while the subsequent container(s) may contain single dose(s) of the same antibody in an amount of 1800 mg and may be designed for subcutaneous administration of the antibody.

### I. Types of Relapsing Multiple Sclerosis

MS, also known as disseminated sclerosis, is a complex disease characterized by considerable heterogeneity in its clinical, pathological, and radiological presentation. It is an autoimmune condition in which the immune system attacks the central nervous system, leading to demyelination (Compston and Coles, Lancet (2008) 372 (9648): 1502-17). MS destroys a fatty layer called the myelin sheath that wraps around and electrically insulates nerve fibers. Almost any neurological symptom can appear with the disease, which often progresses to physical and cognitive disability (Compston and Coles, supra). New symptoms can occur in discrete attacks (relapsing forms), or slowly accumulate over time (progressive forms) (Lublin et al., Neurology (1996) 46 (4): 907-11). Between attacks, symptoms may go away completely (remission), but permanent neurological problems often occur, especially as the disease advances (Lublin et al., supra). Several subtypes, or patterns of progression, have been described, and they are important for prognosis as well as therapeutic decisions. In 1996, the United States National Multiple Sclerosis Society standardized four subtype definitions: relapsing-remitting, secondary progressive, primary progressive, and progressive relapsing (Lublin et al., supra).

Relapsing-remitting multiple sclerosis (RRMS) is characterized by unpredictable acute attacks, called exacerbations or relapses, followed by periods of months to years of relative quiet (remission) with no new signs of disease activity. This describes the initial course of most individuals with MS. RRMS is the most heterogeneous and complex phenotype of the disease, characterized by different levels of disease activity and severity, particularly in the early stages. Inflammation is predominant but neurodegeneration also occurs. Demyelination occurs during acute relapses lasting days to months, followed by partial or complete recovery during the periods of remission. RRMS affects about 65-70% of the MS population and tends to progress to secondary progressive MS.

Secondary progressive MS (SPMS) begins with a relapsing-remitting course, but subsequently evolves into progressive neurologic decline between acute attacks without any definite periods of remission, even though occasional relapses, minor remissions or plateaus may appear. Prior to the availability of approved disease-modifying therapies, data from natural history studies of MS demonstrated that half of RRMS patients would transition to SPMS within 10 years and 90% within 25 years. SPMS affects approximately 20-25% of all people with MS.

The primary progressive subtype (PPMS) is characterized by a gradual but steady progression of disability with no obvious remission after the initial MS symptoms appear (Miller et al., Lancet Neurol (2007) 6 (10): 903-12). It is characterized by progression of disability from onset, with occasional temporary minor improvements or plateaus. A small percentage of PPMS patients may experience relapses. Approximately 10% of all individuals with MS have PPMS. The age of onset for the primary progressive subtype is usually later than other subtypes (Miller et al., supra). Males and females are equally affected.

Progressive relapsing MS (PRMS) is characterized by a steady neurological decline with acute attacks that may or may not be followed by some recovery. This is the least common of all the subtypes described hereinabove.

Cases with non-standard behavior have also been described, sometimes referred to as borderline forms of MS (Fontaine, Rev Neurol. (Paris) (2001) 157 (8-9 Pt 2): 929-34). These forms include Devic's disease, Balo concentric sclerosis, Schilder's diffuse sclerosis, and Marburg multiple sclerosis (Capello et al., Neurol Sci. (2004) 25 Suppl 4:S361-3; Hainfellner et al., J Neurol Neurosurg Psychiatr. (1992) 55 (12): 1194-6).

In some embodiments, the phrase “relapsing MS” or “relapsing forms of MS” (RMS) refers to three different patient subtypes: RRMS, SPMS with relapses, and a clinically isolated demyelination event with evidence of dissemination of lesions in time and space on MRI (see, e.g., European Medicines Agency, Committee for Medicinal Products for Human Use, “Guideline on Clinical Investigation of Medicinal Products for the Treatment of Multiple Sclerosis” (Rev. 2, 2015)).

MS relapse is defined herein as a monophasic, acute, or subacute onset of new neurological symptoms or worsening of previous neurological symptoms with an objective change on neurological examination. In some embodiments, symptoms must:
a) be attributable to MS,
b) last for ≥24 hours, with or without recovery,
c) be present at normal body temperature (i.e., no infection, excessive exercise, or excessively high ambient temperature),
d) be preceded by ≥30 days of clinical stability (including no previous MS relapse); or
e) any combination of a)-d).

In particular embodiments, symptoms of an MS relapse include all of a)-d).

### II. Patient Selection

The present invention relates to treatment of MS patients, e.g., with frexalimab or a related anti-CD40L antibody, such as an anti-CD40L antibody described herein. MS patients in the context of this invention are those who have been diagnosed as having a form of MS by, for example, the history of their symptoms and neurological examination with the help of tests such as magnetic resonance imaging (MRI), spinal taps, evoked potential tests, and laboratory analysis of blood samples. In some embodiments, a patient treated by a therapy described herein has relapsing MS (RMS), such as relapsing-remitting MS or secondary progressive MS with relapses. In certain embodiments, the patient has been diagnosed with RMS according to the 2017 revision of the McDonald diagnostic criteria.

In some embodiments, the patient fulfills any combination of the inclusion and exclusion criteria listed in Example 1. In certain embodiments, the patient fulfills all of the inclusion and exclusion criteria listed in Example 1.

In some embodiments, the patient:
a) is an adult (e.g., is over 18 years of age, or between 18 and 55 years of age, inclusive);
b) has had at least one or at least two documented relapses within the past one to two years (e.g., at least one documented relapse within the previous year, or at least two documented relapses within the previous two years);
c) has body weight within 45-120 kg (inclusive) and body mass index (BMI) within the range of 18.0 to 35.0 kg/m²; or
d) any combination of a)-c).

In certain embodiments, the patient fulfills one, two, or all of criteria a)-c).

In some embodiments, the patient:
e) has not been diagnosed with PPMS or with non-relapsing SPMS (e.g., according to the 2017 revision of the McDonald diagnostic criteria);
f) does not have a history of T lymphocyte or T lymphocyte receptor vaccination, transplantation (including solid organ, stem cell, and bone marrow transplantation) and/or antirejection therapy;
g) does not have a history of diagnosis of progressive multifocal leukoencephalopathy (PML) or evidence of MRI findings suggestive of PML;
h) does not have serious systemic viral, bacterial or fungal infection (e.g., pneumonia, pyelonephritis); infection requiring hospitalization or IV antibiotics; or significant chronic viral, bacterial, or fungal infection (e.g., osteomyelitis) (e.g., within 30 days prior to and during treatment);
i) does not have a history of invasive opportunistic infections, such as, but not limited to, histoplasmosis, listeriosis, coccidioidomycosis, candidiasis, Pneumocystis jirovecii, and aspergillosis;
j) does not have symptomatic herpes zoster (e.g., within three months prior to treatment),
k) does not show evidence of active or latent tuberculosis (TB), e.g., as documented by medical history and examination, chest X-rays (posterior anterior and lateral), and/or TB testing (e.g., either a positive tuberculin skin test (TST; defined as a skin induration <5 mm at 48 to 72 hours, regardless of Bacillus Calmette-Guerin [BCG] or other vaccination history) or a positive (not indeterminate) QuantiFERON®-TB Gold test);
l) does not have a history of malignancy (e.g., within 10 years prior to treatment), except in some embodiments effectively treated carcinoma in situ of the cervix or adequately treated nonmetastatic squamous or basal cell carcinoma of the skin;
m) does not have a history of alcohol or drug abuse (e.g., within one year prior to treatment);
n) does not have a history of any psychiatric disease, behavioral condition, or depression requiring hospitalization (e.g., within two years prior to treatment);
o) does not have a current or chronic history of liver disease, or known hepatic or biliary abnormalities (with the exception of Gilbert's syndrome or asymptomatic gallstones);
p) does not have a history of, or demonstrate clinical evidence, suspicion or significant risk for thromboembolic events, as well as myocardial infarction, stroke and/or antiphospholipid syndrome and does not require antithrombotic treatment;
q) does not have allergies to humanized monoclonal antibodies or severe post-treatment hypersensitivity reactions other than localized injection site reaction, to any biological molecule;
r) has not received any of the following medications, or has not received any of them, e.g., within the listed period prior to treatment;

| Medication | Period Prior to Treatment |
|---|---|
| Systemic corticosteroids, adrenocorticotropic hormone | 1 month |
| Dimethyl fumarate | 1 month |
| Intravenous (IV) immunoglobulin, plasmapheresis, | 2 months |
| fingolimod, natalizumab (participants who have | |
| discontinued natalizumab in the 6 months prior to | |
| randomization should be evaluated to rule out PML) | |
| Teriflunomide | 3 months (unless accelerated |
| | elimination procedure is done) |
| B-cell-depleting therapies such as ocrelizumab and | 6 months (or until return of B-cell |
| rituximab | counts to normal levels, whichever is |
| | longer) |
| Mildly to moderately | 6 months |
| immunosuppressive/chemotherapeutic medications such | |
| azathioprine and methotrexate | |
| Highly immunosuppressive/chemotherapeutic | 2 years |
| medications: mitoxantrone up to 120 mg/m² body | |
| surface area, cyclophosphamide, cladribine | |
| Alemtuzumab | 4 years |
| Other MS-disease modifying treatments | 5 half-lives or until end of |
| | pharmacodynamic activity, whichever |
| | is longer |
| Lymphoid irradiation, bone marrow transplantation, | Any time |
| mitoxantrone (with evidence of cardiotoxicity following | |
| treatment, or cumulative lifetime dose >120 mg/m²), | |
| other strongly immunosuppressive treatments with very | |
| long-lasting effects | |
| Any live (attenuated) vaccine (including but not limited | 3 months |
| to varicella zoster, oral polio, and nasal influenza) | |

MRI: magnetic resonance imaging;
MS: multiple sclerosis;
PML: progressive multifocal leukoencephalopathy;
IV: intravenous.

s) does not have an Expanded Disability Status Scale (EDSS) score >5.5 prior to starting treatment;
t) has not had a relapse in the 30 days prior to starting treatment;
u) does not have positive human immunodeficiency virus (HIV) serology (anti-HIV1 and anti-HIV2 antibodies) or a known history of HIV infection, active or in remission;
v) does not show abnormal laboratory test results in levels of alanine aminotransferase (ALT), aspartate aminotransferase (AST), bilirubin (e.g., unless the patient has documented Gilbert syndrome), hemoglobin, lymphocytes, neutrophils, platelets, and/or estimated glomerular filtration rate, e.g., as defined in Example 1, prior to starting treatment;
w) does not demonstrate presence of hepatitis B surface antigen (HBsAg) and anti-hepatitis B core antibodies (anti-HBc Ab) within three months prior to starting treatment;
x) does not show a positive hepatitis C antibody test result (e.g., within three months prior to starting treatment); or
y) any combination of e)-x).

In certain embodiments, the patient fulfills any 1, 2, 3, 4, 5, 6, 7, 8, 9, 10, 11, 12, 13, 14, 15, 16, 17, 18, 19, or all 20 of criteria e)-x). In particular embodiments, the patient fulfills any 1, 2, 3, 4, 5, 6, 7, 8, 9, 10, 11, 12, 13, 14, 15, 16, 17, 18, 19, 20, 21, 22, 23, or all 24 of criteria a)-x).

### III. Treatment of Multiple Sclerosis

The present invention relates to treating MS, e.g., in a patient as described herein, with frexalimab or a related anti-CD40L antibody (e.g., an anti-CD40L antibody described herein). In some embodiments, the treatment is directed to relapsing MS such as relapsing-remitting MS or secondary progressive MS with relapses.

The present therapies can be used as a first line therapy to treat treatment-naïve patients, i.e., those who have not been treated with MS drugs (e.g., any MS drug recited herein). The present therapies can also be used to treat patients who have been treated with MS drugs (e.g., any MS drug recited herein), but these patients may have failed to respond to the previous treatment, or may have since experienced worsening of the disease or renewed disease activity.

The present therapies can be used alone or in combination with other MS drugs. Currently available MS drugs include, for example, oral drugs such as Aubagio® (teriflunomide), Gilenya® (fingolimod), Tecfidera® (dimethyl fumarate), Vumerity® (diroximel fumarate), Bafiertam® (monomethyl fumarate), Mayzent® (siponimod), Zeposia® (ozanimod), Ponvory® (ponesimod), and Mavenclad® (cladribine); infusion drugs such as Lemtrada® (alemtuzumab), Tysabri® (natalizumab), and Ocrevus® (ocrelizumab); and injectables such as Rebif® (interferon-beta 1a), Plegridy® (pegylated interferon-beta 1a), Copaxone® (glatiramer acetate), and Zinbryta® (daclizumab).

Administration of the anti-CD40L antibody may be, e.g., intravenous or subcutaneous. In some embodiments, the antibody is intravenously administered at a dose of 1000-2000 mg, e.g., about 1000, 1100, 1200, 1300, 1400, 1500, 1600, 1700, 1800, 1900, or 2000 mg, e.g., about every 1, 2, 3, 4, 5, 6, 7, or 8 weeks. In certain embodiments, the dose is 1200 mg. In certain embodiments, the antibody is administered about every four weeks or about every month. In some embodiments, the antibody is subcutaneously administered at a dose of 1800 mg, e.g., about every 4 weeks or about every month. Each SC dose may be given to the patient in a single injection or in multiple injections.

In some embodiments, the anti-CD40L antibody may be intravenously administered to the MS patient at two or more doses (e.g., doses recited above). For example, the first administration of the antibody may be at a first dose (e.g., a loading dose) and subsequent administration of the antibody may be at a second dose. In certain embodiments, the first dose is higher than the second dose. In certain embodiments, the patient is intravenously administered a first antibody dose of 1800 mg followed by subsequent dose(s) of 1200 mg. In particular embodiments, the interval between the doses is about four weeks or about one month.

In some embodiments, the anti-CD40L antibody may be administered to the MS patient using two or more routes of administration. For example, the first administration of the antibody may use a first route of administration (e.g., IV), and subsequent administration of the antibody may use other route(s) of administration (e.g., SC). In certain embodiments, the patient is administered an antibody dose of 1800 mg, wherein the first administration is intravenous and subsequent administration is subcutaneous. In particular embodiments, the interval between the doses is about four weeks or about one month.

The anti-CD40L antibody therapy of the invention is contemplated to be efficacious in RMS patients. The therapy may, e.g., decrease inflammatory activity in the patient. Efficacy can be indicated by measures such as, e.g., reduction in new or total gadolinium-enhancing (GdE) T1 lesions, reduction in new or enlarging T2 lesions, or any combination thereof. Efficacy may also be indicated by measures such as, e.g., reduction in the annual relapse rate (ARR) and/or the time to relapse; delay in progression of disability; improvements in disability, relapses, MRI-derived parameters, neurological rating scales, measures of cognitive impairment, fatigue scales, ambulatory index, and clinical global impression of change as assessed by patient and physicians; as well as absence of disease activity (e.g., absence of MRI activity, relapses and progression). Means for measuring efficacy may include, e.g., Expanded Disability Status Scale (EDSS) evaluation and patient-reported outcomes (e.g., using Multiple Sclerosis Impact Scale (MSIS)-29, Patient Reported Outcome Measurement Information System (PROMIS)—Fatigue-MS-8, Patient's Qualitative Assessment of Treatment Version 3 (PQATv3), and/or Patient Global Impression of Change and Severity Scale (PGIC-Fatigue and PGIS-Fatigue)).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any treatment method disclosed herein may be used to treat any individual as defined herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Frexalimab Clinical Study

Frexalimab was investigated as an MS therapeutic in a randomized, double-blind, placebo-controlled clinical study. Two regimens of administration of frexalimab were tested in men and women aged 18-55 years with relapsing MS (including relapsing-remitting MS and secondary progressive MS with relapses). The study duration was 12 weeks, preceded by a screening period starting not earlier than four weeks before Day 1. The primary objective of the study was to determine the efficacy of frexalimab as measured by reduction of the number of new active brain lesions. Secondary objectives were to evaluate efficacy of frexalimab on disease activity as assessed by other MRI measures, and to evaluate the safety and tolerability of frexalimab in participants with RMS.

The primary endpoint was the number of new gadolinium (Gd)-enhancing T1-hyperintense (GdE T1) lesions at Week 12 (relative to Week 8) as measured by brain magnetic resonance imaging (MRI). This radiographic outcome has been established as a highly reliable predictive biomarker for clinical efficacy in pivotal studies in MS and has been demonstrated to be a predictive biomarker for clinical efficacy (reduction in annualized relapse rate [ARR]) in studies with other MS treatments.

Secondary endpoints to determine the potential benefit of frexalimab in neuroinflammation included the number of new or enlarging T2 lesions at Week 12 (relative to Week 8) and the total number of GdE T1 lesions (at Week 12), as measured by brain MRI.

Another secondary endpoint was evaluation of the safety and tolerability of frexalimab by analyzing criteria such as the number of adverse events and serious adverse events; potentially clinically significant abnormalities in laboratory tests, electrocardiograms, and vital signs; and anti-drug antibodies.

A final secondary endpoint was evaluation of the pharmacokinetics of frexalimab, such as frexalimab plasma concentrations over time and pharmacokinetic parameters.

Exploratory assessments included advanced MRI methods.

A summary of primary, secondary, and exploratory objectives and endpoints for the clinical study is presented in Table 1 below:

TABLE 1
Clinical Study Objectives and Endpoints

| Objectives | Endpoints |
|---|---|
| Primary | |
| To determine the efficacy | Number of new gadolinium (Gd)-enhancing T1-hyperintense (GdE T1) |
| of frexalimab as measured | lesions at Week 12 as measured by brain magnetic resonance |
| by reduction of the number | imaging (MRI) |
| of new active brain lesions | |
| Secondary | |
| To evaluate efficacy of | Number of new or enlarging T2 lesions at Week 12 |
| frexalimab on disease | Total number of GdE T1 lesions at Week 12 |
| activity as assessed by | |
| other MRI measures | |
| To evaluate the safety and | Adverse events (AEs), serious adverse events (SAEs), potentially |
| tolerability of frexalimab | clinically significant abnormalities (PCSAs) in laboratory tests, |
| | electrocardiogram (ECG), and vital signs during the study period |
| | Anti-drug antibodies (ADAs) |
| To evaluate | Frexalimab plasma concentrations over time. Pharmacokinetic (PK) |
| pharmacokinetics of | parameters (maximum concentration [Cₘₐₓ], time to Cₘₐₓ [tₘₐₓ], area |
| frexalimab | under the curve over the dosing interval [AUC₀₋ₜₐᵤ], and elimination |
| | half-life [t_{1/2}]) |
| Exploratory | |
| To evaluate efficacy of | Change from baseline over time in volume, number, and intensity (T1) |
| frexalimab on disease | of slowly evolving lesions (SEL) |
| activity, assessed by | Change in number of phase rim lesions in susceptibility weighted |
| clinical, imaging measures, | imaging (SWI) MRI from baseline over time (subset of centers with |
| and patient-reported | capacity of 3 Tesla MRI) |
| outcomes | Change in magnetization transfer imaging (MTR) of GdE T1 lesions |
| | from Week 8 over time (selected centers) |
| | Number of new GdE T1 lesions over time |
| | Change in volume of T2 lesions from baseline over time |
| | Change in brain volume, including regional changes, from baseline |
| | over time |
| | Change in total number of T1-hypointense lesions from baseline over |
| | time |
| | Proportion of participants with no new MRI disease activity at the end |
| | of 12 weeks of treatment and over time |
| | Number of new or enlarging T2 lesions over time |
| | Number of relapses (annualized relapse rate) over 12 weeks of |
| | treatment and over time |
| | Proportion of relapse-free participants at the end of 12 weeks of |
| | treatment and over time |
| | Change in EDSS from baseline over time |
| | Change in MSIS-29 physical and psychological domains scoring from |
| | baseline over time |
| | Change in PROMIS-Fatigue-MS-8 scoring from baseline over time |
| | Descriptive summaries of PQATv3 scores by treatment arm at |
| | Week 24 |
| | Change in PGIC-Fatigue from baseline over time |
| | Change in PGIS-Fatigue from baseline over time |

In this study, a total of 129 participants were randomly assigned into four cohorts. This Example discusses the treatment regimens and results for the first and third cohorts. The first cohort (52 participants) received an intravenous (IV) dose of frexalimab every four weeks (Q4W), with a loading dose of 1800 mg on Day 1 followed by 1200 mg doses at Weeks 4 and 8. The third cohort (12 participants) received IV placebo Q4W. Unless otherwise indicated, references in the Example to the “frexalimab group” or “frexalimab treatment group” indicate the first cohort, while references to the “placebo group” refer to the third cohort. The second and fourth cohorts relate to another treatment regimen and its placebo arm. The treatment regimens and results for those cohorts are not discussed herein.

An overview of the study interventions for cohorts 1 and 3 is shown below in Table 2. The cohort 1 and 3 participants were switched to open-label frexalimab treatment after Week 12, with an expected treatment duration of 8-76 weeks. Participants from the frexalimab treatment arm continued their previous IV treatment. Participants from the placebo arm received frexalimab IV Q4W (1800 mg loading dose followed by subsequent 1200 mg doses), starting from the Week 12 visit.

TABLE 2
Overview of Study Interventions Administered

| Intervention | | |
|---|---|---|
| name | frexalimab | placebo |
| Type | Drug | Drug |
| Dose | 300 mg/2 mL vial | 2 mL vial |
| formulation | | (identical to |
| | | frexalimab in |
| | | appearance) |
| Unit dose | 300 mg/2 mL | 2 mL |
| strength(s) | | |
| Dosage level(s) | 1800 mg loading | Not applicable |
| | dose; then 1200 | |
| | mg q4w | |
| Route of | IV infusion | IV infusion |
| administration | | |

The composition of the frexalimab drug product solution included frexalimab, L-histidine HCl, L-histidine, L-arginine HCl, sucrose, polysorbate-80, and water for injection. The pH of the formulated solution was 5.6.

The demographic characteristics at baseline were similar across both the treatment and placebo cohorts, although the participants in the placebo group were younger and had a higher total number of GdE T1 and T2 lesions at baseline compared to the participants in the frexalimab treatment group. Of the participants, 50 in the first cohort and 11 in the third cohort had relapsing-remitting multiple sclerosis. 2 in the first cohort and 1 in the third cohort had secondary progressive multiple sclerosis with relapses.

The following inclusion and exclusion criteria were used in selecting patients eligible to participate in the study:

### Inclusion Criteria

Participant must be 18 to 55 years of age inclusive, at the time of signing the informed consent.
The participant must have been diagnosed with RMS (relapsing-remitting MS or secondary progressive MS participants with relapses) according to the 2017 revision of the McDonald diagnostic criteria.
The participant must have at least 1 documented relapse within the previous year, or ≥2 documented relapses within the previous 2 years, or ≥1 active Gd-enhancing brain lesion on an MRI scan in the past 6 months and prior to screening.
Body weight within 45 to 120 kg (inclusive) and body mass index (BMI) within the range 18.0 to 35.0 kg/m² (inclusive) at Screening.

### Exclusion Criteria

The participant has been diagnosed with PPMS according to the 2017 revision of the McDonald diagnostic criteria or with non-relapsing SPMS.
The participant has conditions or situations that would adversely affect participation in this study, including but not limited to:
   A short life expectancy due to preexisting health condition(s) as determined by judgment of investigator or their treating physician.
   Medical condition(s) or concomitant disease(s) making them nonevaluable for the primary efficacy endpoint or that would adversely affect participation in this study, as judged by the Investigator.
   A requirement for concomitant treatment that could bias the primary evaluation by judgment of investigator.
   Contraindication for MRI, ie, presence of pacemaker, metallic implants in high-risk areas (ie, artificial heart valves, aneurysm/vessel clips), presence of metallic material (eg, shrapnel) in high risk areas, known history of allergy to any contrast medium, or history of claustrophobia that would prevent completion of all protocol scheduled MRI.
   Contraindications to use MRI Gd contrast-enhancing preparations.
The participant has a history of or currently has concomitant medical or clinical conditions that would adversely affect participation in this study, including but not limited to:
   A history of T-lymphocyte or T-lymphocyte-receptor vaccination, transplantation (including solid organ, stem cell, and bone marrow transplantation) and/or antirejection therapy.
   A history of diagnosis of progressive multifocal leukoencephalopathy (PML) or evidence of findings suggestive of PML on the baseline MRI.
   Serious systemic viral, bacterial or fungal infection (eg, pneumonia, pyelonephritis), infection requiring hospitalization or IV antibiotics or significant chronic viral, bacterial, or fungal infection (eg, osteomyelitis) 30 days before and during screening.
   Participants with a history of invasive opportunistic infections, such as, but not limited to histoplasmosis, listeriosis, coccidioidomycosis, candidiasis, Pneumocystis jirovecii, and aspergillosis, regardless of resolution.
   Symptomatic herpes zoster within 3 months prior to screening.
   Evidence of active or latent tuberculosis (TB) as documented by medical history and examination, chest X-rays (posterior anterior and lateral), and TB testing: either a positive tuberculin skin test (TST; defined as a skin induration <5 mm at 48 to 72 hours, regardless of Bacillus Calmette-Guerin [BCG] or other vaccination history) or a positive (not indeterminate) QuantiFERON®-TB Gold test.
   Any other active infections that would adversely affect participation or IMP administration in this study, as judged by the Investigator.
   A history of malignancy within 10 years prior to the first screening visit, except effectively treated carcinoma in situ of the cervix or adequately treated nonmetastatic squamous or basal cell carcinoma of the skin.
   A history of alcohol or drug abuse within 1 year prior to the first screening visit.
   A history of any psychiatric disease, behavioral condition, or depression requiring hospitalization within 2 years prior to the first screening visit.
   Presence of any screening laboratory or ECG values outside normal limits that are considered in the Investigator's judgment to be clinically significant.
   Current or chronic history of liver disease, or known hepatic or biliary abnormalities (with the exception of Gilbert's syndrome or asymptomatic gallstones).
History, clinical evidence, suspicion or significant risk for thromboembolic events, as well as myocardial infarction, stroke and/or antiphospholipid syndrome and any participants requiring antithrombotic treatment.
Allergies to humanized monoclonal antibodies or severe post-treatment hypersensitivity reactions other than localized injection site reaction, to any biological molecule (including, but not limited to, erythema multiforme major, linear IgA dermatosis, toxic epidermal necrolysis, and exfoliative dermatitis).
Prior/concomitant therapy
   The participant has received any of the following medications/treatments within the specified time frame before any baseline assessment (no wash-out is required for interferons beta or glatiramer acetate treatments):

| | Exclusionary if used/used within |
|---|---|
| Medication | required wash-out period |
| Systemic corticosteroids, adrenocorticotropic hormone | 1 month prior to screening MRI scan |
| Dimethyl fumarate | 1 month prior to randomization |
| Intravenous (IV) immunoglobulin, plasmapheresis, | 2 months prior to randomization |
| fingolimod, natalizumab (participants who have | |
| discontinued natalizumab in the 6 months prior to | |
| randomization should be evaluated to rule out PML) | |
| Teriflunomide | 3 months prior to randomization. No |
| | time restriction if accelerated |
| | elimination procedure is done |
| B-cell-depleting therapies such as ocrelizumab and | 6 months prior to randomization or |
| rituximab | until return of B-cell counts to normal |
| | levels, whichever is longer |
| Mildly to moderately | 6 months prior to randomization |
| immunosuppressive/chemotherapeutic medications such | |
| azathioprine and methotrexate | |
| Highly immunosuppressive/chemotherapeutic | 2 years prior to randomization |
| medications: mitoxantrone up to 120 mg/m² body | |
| surface area, cyclophosphamide, cladribine | |
| Alemtuzumab | 4 years prior to randomization |
| Other MS-disease modifying treatments | 5 half-lives or until end of |
| | pharmacodynamics activity, whichever |
| | is longer |
| Lymphoid irradiation, bone marrow transplantation, | Any time (not eligible for enrollment) |
| mitoxantrone (with evidence of cardiotoxicity following | |
| treatment, or cumulative lifetime dose > 120 mg/m²), | |
| other strongly immunosuppressive treatments with very | |
| long-lasting effects | |
| Any live (attenuated) vaccine (including but not limited | 3 months prior to randomization |
| to varicella zoster, oral polio, and nasal influenza) | |

MRI: magnetic resonance imaging;
MS: multiple sclerosis;
PML: progressive multifocal leukoencephalopathy;
IV: intravenous.
teriflunomide accelerated elimination procedure must be followed per locally approved product information.

Prior/concurrent clinical study experience
   The participant has taken other investigational drug(s) within 3 months or 5-half-lives, whichever is longer, before the screening visit.
Diagnostic assessments
   The participant has an EDSS score >5.5 at the first screening visit.
      The participant has had a relapse in the 30 days prior to randomization.
   Positive human immunodeficiency virus (HIV) serology (anti-HIV1 and anti-HIV2 antibodies) or a known history of HIV infection, active or in remission.
   Abnormal laboratory test(s) at Screening:
   Alanine aminotransferase (ALT) or aspartate aminotransferase (AST)>2.0×ULN
   Bilirubin >1.5×ULN; unless the participant has documented Gilbert syndrome (isolated bilirubin >1.5×ULN is acceptable if bilirubin is fractionated and direct bilirubin <35%)
   Hemoglobin <11 g/100 mL for males and <10 g/100 mL for females
   Lymphocytes <1000/mm3
   Neutrophils <1500/mm3 (except <1000/mm3 for participants of African descent)
   Platelets <140 000/mm3
   Estimated Glomerular filtration rate <60 mL/min/1.73 m2 (Modification of Diet in Renal Disease [MDRD])
   Presence of hepatitis B surface antigen (HBsAg) and anti-hepatitis B core antibodies (anti-HBc Ab) at screening or within 3 months prior to first dose of study intervention. If anti-HBs negative and anti-HBc positive: perform hepatitis B virus DNA test to confirm.
   Positive hepatitis C antibody test result at screening or within 3 months prior to starting study intervention. NOTE: Participants with positive Hepatitis C antibody due to prior resolved disease can be enrolled, only if a confirmatory negative Hepatitis C RNA test is obtained.

### A. Efficacy Results

### Primary Efficacy Endpoint:

The study demonstrated a reduction in the number of new active GdE T1 lesions after 12 weeks (relative to Week 8) as detected by brain MRI for the frexalimab treatment group as compared to the placebo group. An 89% reduction (62%-97%) in the adjusted mean monthly count of new GdE T1 lesions in the frexalimab group as compared to the placebo group was observed. See Table 3 below:

TABLE 3
Adjusted Mean and Rate Ratio Estimates (95% CI) of
New Gd-Enhancing T1-Hyperintense (GdE T1)
Lesions Monthly Count at Week 12

| New GdE T1 lesion monthly count at | PLACEBO^{a} | FRX 1200 |
|---|---|---|
| Week 12: Statistics | (N = 22) | (N = 47) |
| Estimated mean of new GdE T1 lesion | | |
| monthly count | | |
| Adjusted mean (SE)^{b} | 1.4 (0.54) | 0.2 (0.07) |
| (95% CI) | (0.62 to 2.98) | (0.06 to 0.39) |
| Adjusted rate ratio (SE)^{b} | | 0.11 (0.07) |
| (95% CI) | | (0.03 to 0.38) |

^{a}Includes all placebo arms
^{b}Based on a negative binomial regression model adjusting for the categorical baseline GdE T1 lesion count (presence/absence) as covariate, treatment as factor. The log of the duration (in months) between the Week 12 MRI and previous MRI was taken into account as an offset variable
FRX: frexalimab

Reduction in new GdE T1 lesions continued to at least 24 weeks for the frexalimab treatment group. Lesion counts were numerically very low in participants receiving the frexalimab treatment (n=37/38 with no new GdE lesions).

### Secondary Efficacy Endpoints:

The results of the two main secondary endpoints were consistent with the results of the primary analysis, with the frexalimab treatment group showing a reduction of new or enlarging T2 lesions and a reduction in the total number of GdE T1 lesions over time as compared to placebo. See Tables 4 and 5 below:

TABLE 4
Adjusted Mean and Rate Ratio Estimates (95% CI) of
New or Enlarging T2 Lesions Monthly Count at Week 12

| New or enlarging T2 lesion monthly | PLACEBO^{a} | FRX 1200 |
|---|---|---|
| count at Week 12: Statistics | (N = 22) | (N = 47) |
| Estimated mean of new or enlarging T2 | | |
| lesion monthly count | | |
| Adjusted mean (SE)^{b} | 3.5 (1.44) | 0.3 (0.12) |
| (95% CI) | (1.59 to 7.86) | (0.14 to 0.65) |
| Adjusted rate ratio (SE)^{b} | | 0.08 (0.05) |
| (95% CI) | | (0.03 to 0.26) |

^{a}Includes all placebo arms
^{b}Based on a negative binomial regression model adjusting for the baseline T2 lesion count, and treatment as factor.
The log of the duration (in months) between the Week 12 MRI and previous MRI was taken into account as an offset variable
FRX: frexalimab

TABLE 5
Adjusted Mean and Rate Ratio Estimates (95% CI) of
the Total Number of GdE T1 Lesions at Week 12

| Total number of GdE T1 lesions at | PLACEBO^{a} | FRX 1200 |
|---|---|---|
| Week 12: Statistics | (N = 22) | (N = 47) |
| Estimated mean of total number of GdE | | |
| T1 lesion | | |
| Adjusted mean of total number of GdE | 1.7 (0.67) | 0.2 (0.09) |
| T1 lesion monthly count (SE)^{b} | (0.80 to 3.68) | (0.09 to 0.46) |
| (95% CI) | | |
| Adjusted rate ratio (SE)^{b} | | 0.12 (0.07) |
| (95% CI) | | (0.04 to 0.36) |

^{a}Includes all placebo arms
^{b}Based on a negative binomial regression model adjusting for the categorical baseline GdE T1 lesion count (presence/absence), and treatment as factor.
FRX: frexalimab

### B. Safety Results

No participants had treatment-emergent adverse events (TEAE) leading to death, treatment-emergent serious adverse events (SAE) s, or severe TEAEs. See Table 6 below. The most common adverse events (≥4% in the frexalimab treatment group) were COVID-19 and headache.

TABLE 6
Overview of Adverse Event Profile: Treatment Emergent Adverse Events

| | PLACEBO | FRX 1200 |
|---|---|---|
| n (%) | (N = 12) | (N = 52) |
| Participants with any TEAE | 3 (25.0) | 15 (28.8) |
| Participants with any treatment | 0 | 0 |
| emergent SAE | | |
| Participants with any TEAE leading to | 0 | 0 |
| death | | |
| Participants with any TEAE leading to | 0 | 0 |
| permanent treatment discontinuation | | |
| Participants with any treatment | 0 | 1 (1.9) |
| emergent AESI | | |

TEAE: Treatment emergent adverse event,
SAE: Serious adverse event,
AESI: Adverse event of special interest
n (%) = number and percentage of participants with at least one TEAE
FRX: frexalimab

### C. Pharmacokinetic Results

Pharmacokinetic results obtained from the clinical study are shown in Table 7 below:

TABLE 7
Mean (CV %) of Frexalimab Plasma Exposure Parameters

| | After first dose | | | After last dose | | | Over last |
|---|---|---|---|---|---|---|---|
| | (Loading Dose) | | | (W 8) | | | 4 weeks |
| | Cₘₐₓ | AUCₜₐᵤ | C_{trough} | C_{trough} | Cₘₐₓ | AUCₜₐᵤ | AUC_{W 8-W 12} |
| | (μg/mL) | (μg · h/mL) | (μg/mL) | (μg/mL) | (μg/mL) | (μg · h/mL) | (μg · h/mL) |
| FRX n | 52 | 52 | 52 | 51 | 51 | 51 | 51 |
| | 623 (20.8) | 158000 (5.6) | 139 (25.3) | 166 (9.7) | 579 (20.5) | 190000 (23.5) | 190000 (23.5) |

tau = interval between dosing (4 weeks)
FRX: frexalimab

### D. Conclusions

Frexalimab, administered according to the above-described regimen, showed efficacy in treating relapsing MS, reducing the number of new GdE T1 lesions at 12 weeks by 89%. The regimen was well tolerated and demonstrated an acceptable safety profile. No safety concerns emerged from the analysis of the double-blind period of this study.

1-18. (canceled)

19. A method of treating relapsing multiple sclerosis (MS) in a human patient in need thereof, comprising intravenously administering to the patient a monoclonal anti-human CD40L antibody, wherein the heavy chain CDR1-3 and light chain CDR1-3 of said antibody comprise SEQ ID NOs: 1-6, respectively, and wherein the antibody is administered to the patient at a dose of 1000-2000 mg about every four weeks or about every month.

20. The method of claim 19, wherein the antibody comprises a heavy chain variable domain and a light chain variable domain comprising SEQ ID NOs: 7 and 8, respectively.

21. The method of claim 19, wherein the antibody comprises a heavy chain and a light chain comprising SEQ ID NOs: 9 and 10, respectively.

22. The method of claim 19, wherein the dose is 1200 mg.

23. The method of claim 19, wherein the first administration of the antibody is at a first dose, and subsequent administration of the antibody is at a second dose, wherein the first dose is higher than the second dose.

24. The method of claim 23, wherein
a) the first dose is 1800 mg,
b) the second dose is 1200 mg, or
c) a) and b).

25. The method of claim 19, wherein the patient has relapsing-remitting MS.

26. The method of claim 19, wherein the patient has secondary progressive MS with relapses.

27. A method of treating relapsing multiple sclerosis (MS) in a human patient in need thereof, comprising administering to the patient a monoclonal anti-human CD40L antibody, wherein the heavy chain CDR1-3 and light chain CDR1-3 of said antibody comprise SEQ ID NOs: 1-6, respectively, and wherein the antibody is administered to the patient at a dose of 1800 mg about every four weeks or about every month.

28. The method of claim 27, wherein the antibody comprises a heavy chain variable domain and a light chain variable domain comprising SEQ ID NOs: 7 and 8, respectively.

29. The method of claim 27, wherein the antibody comprises a heavy chain and a light chain comprising SEQ ID NOs: 9 and 10, respectively.

30. The method of claim 27, wherein the antibody is administered subcutaneously.

31. The method of claim 27, wherein the first administration of the antibody is intravenous and subsequent administration of the antibody is subcutaneous.

32. The method of claim 27, wherein the patient has relapsing-remitting MS.

33. The method of claim 27, wherein the patient has secondary progressive MS with relapses.

34. A method of treating relapsing multiple sclerosis (MS) in a human patient in need thereof, comprising administering to the patient a monoclonal anti-human CD40L antibody that comprises a heavy chain and a light chain comprising SEQ ID NOs: 9 and 10, respectively, wherein
a) the antibody is intravenously administered at a first dose of 1800 mg and at subsequent doses of 1200 mg; or
b) the antibody is administered at a dose of 1800 mg, wherein the first dose is administered intravenously and subsequent doses are administered subcutaneously; and
wherein the interval between doses is about four weeks or about one month.

35. The method of claim 34, wherein the patient has relapsing-remitting MS.

36. The method of claim 34, wherein the patient has secondary progressive MS with relapses.
